(19) United States
(12) Patent Application Publication
(10) Pub. No.: US 2002/0016468 A1
(43) Pub. Date: Feb. 7, 2002

(54) PESTICIDAL 1-ARYLPYRAZOLE DERIVATIVES

(75) Inventors: DAVID TREADWAY MANNING, CARY, NC (US); MICHAEL PILATO, CARY, NC (US); TAI-TEH WU, CHAPEL HILL, NC (US); DAVID WILLIAM HAWKINS, ESSEX (GB)

Correspondence Address: MICHELE C BOSCH, FINNEGAN HENDERSON FARABOW GARRETT & DUNNER, 1300 I STREET N W, WASHINGTON, DC, 20005-3315, US

(73) Assignee: Rhone-Poulenc Agrochimie, Lyon (FR)

(21) Appl. No.: 09/339,176

(22) Filed: Jun. 24, 1999

(51) Int. Cl. C 07D 4 1/02; A01N 43/40; A01N 43/38; A01N 43/56

(52) U.S. Cl. 546/276.1; 548/367.4; 548/369.4; 548/370.1; 514/414; 514/341; 514/407

(57) ABSTRACT

The invention relates to 1-arylpyrazole-3-thiocarboxamide derivatives of formula (I):

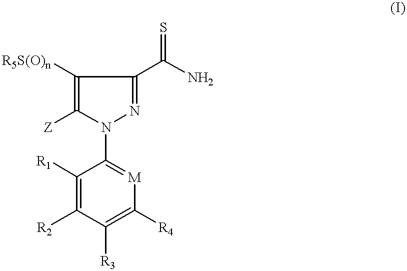

wherein R₁, R₂, R₃, R₄, R₅, M, Z and n are as defined in the description, and to their use as insecticides.

### CROSS-REFERENCE TO RELATED APPLICATIONS

This application is a continuation of International Patent Application No. PCT/EP97/07116, filed Dec. 18, 1997 and designating the United States, which is incorporated by reference herein in its entirety and relied upon, and which claims the priority of United States Provisional Patent Application No. 60/033,885, filed Dec. 24, 1996.

### BACKGROUND OF THE INVENTION

1. Field of the Invention

The invention relates to new 3-thiocarboxamide derivatives of 1-arylpyrazoles and derivatives thereof. The invention further pertains to compositions of said compounds and methods, using said compounds, for the control of arthropod pests, in particular to the application of said compounds or compositions in agricultural methods of use, particularly as pesticides, for controlling arthropods, especially insects by systemic action.

2. Description of the Related Art

The control of insects, nematodes or hehninths using 1-arylpyrazoles has been described in International Patent Publications No. WO 93/06089 (and the equivalent U.S. Pat. No. 5,451,598), WO 94/21606 and WO 87/03781, as well as in European Patent Publication Numbers 0295117, 659745, 679650, 201852 and 412849, German Patent No. DE19511269 and U.S. Pat. No. 5,232,940.

### OBJECTS AND SUMMARY OF THE INVENTION

It is an object of the present invention to provide new pesticidal compounds of the 1-arylpyrazole family together with processes for their preparation.

A second object of the present invention is to provide pesticidal compositions and pesticidal methods of use of the pesticidal pyrazole compounds against arthropods, especially insects, particularly in agricultural or horticultural crops, forestry, veterinary medicine or livestock husbandry, or in public health.

A third object of the present invention is to provide very active compounds, with broad spectrum pesticidal activity, as well as compounds with selective special activity, e.g. aphicidal, miticidal, foliar insecticidal, soil insecticidal, systemic, antifeeding or pesticidal activity via seed treatment.

A fourth object of the present invention is to provide compounds with substantially enhanced and more rapid activity, especially against insects and more particularly insects in their larval stages.

A fifth object of the present invention is to provide compounds with greatly improved (faster and greater) penetration into pest species when topically applied and to thus provide enhanced movement of the compounds to the pesticidal site(s) of action within the pest.

These and other objects of the invention shall become apparent from the description of the present invention which follows. The objects are met in whole or in part by the invention.

The invention provides insecticidal 1-arylpyrazoles having the general formula (I):

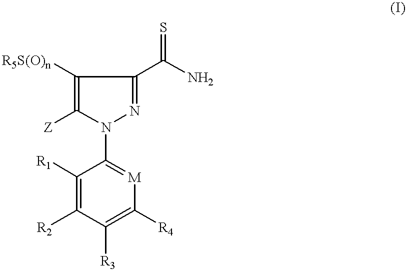

wherein:

R₁ is H or halogen;

each of R₂ and R₄, which are identical or different, is H, halogen or alkyl;

R₃ is halogen, haloalkyl, haloalkoxy or R₁₀S(O)ₘ; preferably, R₃ is halogen, haloalkyl or haloalkoxy;

R₅ is alkyl, haloalkyl, alkenyl and alkynyl; or a cycloalkyl ring containing 3 to 5 carbon atoms; preferably R₅ is alkyl;

Z is hydrogen, halogen, alkyl, formyl, —C(O)alkyl, haloalkyl, alkenyl, hydrazino, alkoxycarbonyl, alkylthiocarbonyl, alkoxyalkylideneamino, 1H-pyrrol-1-yl or 1H-pyrazol-1-yl; or, preferably, Z is amino, R₆NH-or R₇R₈N—;

each of R₆, R₇ and R₈, which are identical or different, is alkyl—S(O)ₚ—, formyl, alkynyl containing from 3 to 6 carbon atoms, alkoxycarbonyl, alkylthiocarbonyl or aroyl; or alkyl, alkenyl containing from 3 to 6 carbon atoms, or —C(O)alkyl wherein the alkyl and alkenyl portions are optionally substituted by one or more R₉;

or R₇ and R₈ are joined so as together form a divalent radical having 4 to 6 atoms in the chain, this divalent radical being alkylene, alkyleneoxyalkylene or alkyleneaminoalkylene, preferably to form a morpholine, pyrrolidine, piperidine or piperazine ring;

R₉ is cyano, nitro, alkoxy, haloalkoxy, R₁₀S(O)_{q}—, —C(O)alkyl, alkoxycarbonyl, aminocarbonyl, alkylaminocarbonyl, dialkylaminocarbonyl, —CO₂H, halogen, hydroxy, aminosulfonyl, alkylaminosulfonyl or dialkylaminosulfonyl;

R₁₀ is lower alkyl or lower haloalkyl;

m, n, p and q are 0, 1 or 2;

M is C-halo, C—CH₃, C—CH₂F, C—CH₂Cl, C—NO₂ or N; and pesticidally active salts thereof. By the term “pesticidally acceptable salts” is meant salts the anions and cations of which are known and accepted in the art for the formation of pesticidally acceptable salts. Preferably such salts are water soluble. Suitable acid addition salts formed from compounds of formula (I) containing an amine group, include salts with inorganic acids for example hydrochlorides, phosphates, sulfates and nitrates, and salts with organic acids for example acetates. Suitable salts with bases formed from compounds of formula (I) containing a carboxylic acid group, include alkali metal (for example sodium or potassium) salts, ammonium salts and organic amine (for example diethanolamine or morpholine) salts.

### DETAILED DESCRIPTION OF THE INVENTION

Unless otherwise specified alkyl, alkoxy and alkylthio groups have from one to six (preferably one to four) carbon atoms. Alkenyl and alkynyl groups have from two to six (preferably two to four) carbon atoms. In the instant invention, some words are used in a specific sense: The term “aminocarbonyl” means a carbamoyl radical, that is, a radical of the formula —C(O)NH₂. Similarly, the term “alkylaminocarbonyl” means an alkylcarbamoyl radical, that is, a radical of the formula —C(O)—NH-alkyl; and the term “dialkylaminocarbonyl” means a dialkylcarbamoyl radical, that is, a radical of the formula —C(O)—N(alkyl)₂ in which the alkyl moieties can be the same or different. The term “aminosulfonyl” means a sulfamoyl radical, that is, -SO₂NH₂. Similarly, the term “alkylaminosulfonyl” means an alkylsulfamoyl radical, that is, a radical of the formula —SO₂NH-alkyl; while the term “dialkylaminosulfonyl” means a dialkylsulfamoyl radical, which has the formula —SO₂N(alkyl)₂ wherein the alkyl moieties can be the same or different.

The term “halo” before the name of a radical means that this radical is partially or completely halogenated, that is to say, substituted by F, Cl, Br, or I, in any combination, preferably by F or Cl. The term “halogen” means F, Cl, Br or I. When the name of any substituent is repeated, it keeps the same meaning unless otherwise specified. The term “aroyl” designates a carbonyl aromatic radical, that is, aryl—C(O)—, which is preferably a benzoyl optionally substituted by one or more alkyl or halogen groups.

A particularly preferred class of compounds of formula (I) are those wherein:

R₁ is F, Cl, Br or H;

R₂ and R₄ are H;

R₃ is —CF₃, —OCF₃, —CHF₂, —S(O)ₘCF₃, —CFCl₂, —CF₂Cl, —OCF₂Cl, —OCFC₂, Cl,

Br or F;

R₅ is methyl or ethyl;

Z is H, halogen (F, Cl, Br), C₁-C₃ alkyl, C₁-C₃ haloalkyl, C₂-C₃ alkenyl; or amino, —NHR₆ or —NR₇R₈, wherein each of R₆, R₇ and R₈, which are identical or different, is C₁-C₃ alkyl, C₃ alkenyl or —C(O)alkyl wherein the alkyl and alkenyl portions are unsubstituted or substituted with cyano, alkoxy, alkyl—S(O)ₚ—, nitro, alkoxycarbonyl, —C(O)alkyl, aminocarbonyl, alkylaminocarbonyl, dialkylarninocarbonyl, —CO ₂H, hydroxy or halogen (F, Cl, Br); and M is CCl, CF, CBr or N.

A further especially preferred class of compounds are those wherein:

R₁ is Cl or Br;

R₂and R₄are H;

R₃ is —CF₃, —OCF₃ or Cl;

R₅ is methyl or ethyl;

Z is H, halogen (F, Cl, Br), C₁-C₃ alkyl, C₁-C₃ haloalkyl, C₂-C₃ alkenyl; or amino, —NHR6 or —NR₇R₈, wherein each of R₆, R₇ and R₈, which are identical or different, is C₁-C₃ alkyl, C₃ alkenyl or —C(O)alkyl wherein the alkyl or alkenyl portions are unsubstituted or substituted with cyano, alkoxy, alkyl—S(O)ₚ, nitro, alkoxycarbonyl, —C(O)alkyl, aminocarbonyl, alkylaminocarbonyl, dialkylaminocarbonyl, —CO ₂H, hydroxy or halogen (F, Cl, Br); and

M is CCl, CBr or N.

A further especially preferred class of compounds are those wherein:

R₁ is Cl or Br;

R₂ and R₄ are H;

R₃ is —CF₃, —OCF₃ or Cl;

R₅ is optionally halogenated methyl or ethyl;

Z is H or amino; and

M is CCl, CBr or N.

A further especially preferred class of compounds are those wherein:

R₁ is Cl or Br;

R₂ and R₄ are H;

R₃ is —CF₃, —OCF₃ or Cl;

R₅ is methyl or ethyl;

Z is H or amino; and

M is CCl, CBr or N.

For the above preferred compounds, there are optimum combinations of substituent groups.

Preferred phenyl groups or pyridyl groups comprising the R₁-R₄ and M radicals in formula (I) are: 2,6-dichloro-4-trifluoromethylphenyl; 2,6-dichloro-4-trifluoromethoxyphenyl; 2-bromo-6-chloro-4-trifluoromethylphenyl; 2-bromo-6-chloro-4-trifluoromethoxyphenyl; 2,6-difluoro-4-trifluoromethylphenyl; 2-chloro-4-trifluoromethylphenyl; 2,6-dichloro-3-methyl-4-trifluoromethylphenyl; 3-chloro-5-trifluoromethyl-2-pyridinyl; 3-chloro-5-trifluoromethoxy-2-pyridinyl; 3,5-dichloro-2-pyridinyl; 2,6-dichloro-4-bromophenyl; 2,4,6-trichlorophenyl; 2-bromo-6-fluoro-4-difluoromethylphenyl; 2-chloro-6-fluoro-4-trifluoromethylphenyl; 2,6-dibromo-4-trifluoromethylphenyl; 2,6-dibromo-4-trifluoromethoxyphenyl; and 2-bromo-4-trifluoromethylphenyl.

Further preferred Z substituents in formula (I) are: acetylamino; amino; 2-n-butoxypropionylamino; methyl; hydroxyacetylamino; ethyl; 3-ethylsulfinylpropylamino; bromo; formylamino; chloro; methylamino; ethylamino; 2-hydroxyethylamino; 2-methoxyethylamino; methylsulfonylamino; 2-ethylsulfonylethylamino; 4-methoxybenzoylamino; 2-cyanoethylamino; 2-methylthioethylamino; 2-aminocarbonylethylamino (2-carbamoylethylamino); 2-methylsulfinylethylamino; 3-methoxycarbonylpropylamino; 2-ethylsulfinylethylamino; 2-methylsulfonylethylamino; cyanomethylamino; 2-ethylthioethylamino; aminocarbonylmethylamino (carbamoylmethylamino); dimethylamino; 2-nitroethylamino; 2-acetylethylamino; methylcarbonylmethylamino (acetylmethylamino); methoxycarbonyl; and ethoxycarbonyl.

Further preferred —S(O)ₙR₅ substituents in formula (I) are: methylthio; methylsulfinyl; methylsulfonyl; ethylsulfinyl; ethylsulfonyl; ethylthio; cyclopropylsulfinyl; cyclopropylthio; cyclopropylsulfonyl; isopropylsulfinyl; isopropylsulfonyl; and isopropylthio.

Particularly preferred pyrazole derivatives include the following, the numbers 1-11 being assigned to these compounds for reference and identification purposes.

1. 5-amino-1-(2,6-dichloro-4-trifluoromethylphenyl)-4-methylthiopyrazole-3-thiocarboxamide

2. 5-amino-1-(2,6-dichloro-4-trifluoromethylphenyl)-4-trifluoromethylthiopyrazole-3-thiocarboxamide

3. 4-dichlorofluoromethylsulfonyl-1-(2,4,6-trichlorophenyl)pyrazole-3-thiocarboxamide

4. 5-amino-1-(2,6-dichloro-4-trifluoromethylphenyl)-4-methylsulfinylpyrazole-3-thiocarboxamide

5. 5-amino-1-(2,6-dichloro-4-trifluoromethylphenyl)-4-methylsulfonylpyrazole-3-thiocarboxamide

6. 5-amino-1-(2-bromo-6-chloro-4-trifluoromethylphenyl)-4-methylsulfinylpyrazole-3-thiocarboxamide

7. 5-amino-1-(2,6-dichloro-4-trifluoromethoxyphenyl)-4-methylsulfinylpyrazole-3-thiocarboxamide

8. 5-amino-1-(2,6-dichloro-4-trifluoromethylphenyl)-4-ethylsulfinylpyrazole-3-thiocarboxamide

9. 5-amino-1-(2-chloro-4-trifluoromethylphenyl)-4-methylsulfinylpyrazole-3-thiocarboxamide

10. 5-amino-1-(2-bromo-6-chloro-4-trifluoromethylphenyl)-4-ethylsulfinylpyrazole-3-thiocarboxamide

11. 5-amino-1-(2,6-dichloro-4-trifluoromethoxyphenyl)-4-ethylsulfinylpyrazole-3-thiocarboxamide.

### METHODS OR PROCESSES OF SYNTHESIS

The compounds of general formula (I) can be prepared by the application or adaptation of known methods (i.e., methods heretofore used or described in the chemical literature including the Chemical Abstracts) employing 3-cyano-1-phenylpyrazoles or 3-cyano-1-(2-pyridinyl)pyrazole intermediates of known formula or can be prepared by methods or processes similar to those described in EP 0295117, EP 0234119, WO 87/03781, EP 0500209 and EP 780378.

According to a feature of this invention compounds of formula (I) may be prepared by the reaction of a compound of formula (II):

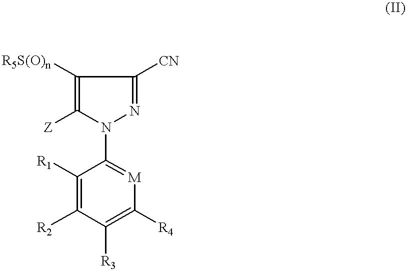

wherein R₁, R₂, R₃, R₄, R₅, M and Z are defined above with an alkali or alkaline earth metal hydrosulfide, such as lithium, potassium, calcium or preferably sodium hydrosulfide, in an inert solvent for example N,N-dimethylformamide, pyridine, dioxan, tetrahydrofuran, sulfolane, dimethyl sulfoxide, methanol or ethanol at a temperature from −35° C. to 50° C. preferably 0° C. to 30° C. Optionally the hydrosulfide may be generated in situ by treatment with H₂S in the presence of an organic base, such as a metal alkoxide or trialkylamine or an inorganic base, such as an alkaline or alkaline earth metal hydroxide or a carbonate, such as sodium, potassium or ammonium carbonate. The use of a metal complexing agent, such as a crown ether, can be of benefit in accelerating the reaction. The reaction of hydrosulfide salt with the 3-cyanopyrazole can also be conducted in a two-phase water/organic solvent system using a phase transfer catalyst such as a crown ether or a tetraalkylammonium salt such as tetra-n-butylammonium bromide or benzyltrimethylammonium chloride. Organic solvents suitable for use in a two-phase system with water include benzene, toluene, dichloromethane, 1-chlorobutane and methyl tertiary-butyl ether.

Compounds of formula (I) may also be prepared from compounds of formula (II) by treatment with the reagent Ph₂PS₂, as described in Tet. Lett., 24 (20), 2059 (1983);

According to a further feature of the invention compounds of general formula (I) wherein R₁, R₂, R₃, R₄, R₅, M and Z are as defined above and in which n represents 1 or 2 may be prepared by the oxidation of the corresponding compounds of formula (I) in which n represents 0 or 1. The reaction should be performed with a mild oxidising agent (to prevent destruction of the thioamide functional group) such as sodium periodate in an inert solvent for example methylene chloride at a temperature from −40° C. to the reflux temperature of the solvent.

According to a further feature of the present invention compounds of general formula (I) wherein Z represents hydrogen, halogen, alkyl, haloalkyl, amino, R₆NH or R₇R₈N- wherein R₆, R₇ and/or R₈ represent alkyl, haloalkyl, —C(O)alkyl, alkoxycarbonyl, formyl and—S(O)ₚalkyl; or R₆ and R₇ are joined so as together form a divalent radical having 4 to 6 atoms in the chain, may be prepared by methods described in one or more of International Publications No. WO 94/21606, WO 93/06089 and WO 87/03781, European Patent Publication No. 0295117 and EP 511845, Hatton et al U.S. Pat. No. 5,232,940, and German Patent Publication No. DE 19511269.

According to a further feature of the invention compounds of general formula (I) wherein R₁, R₂, R₃, R₄, R₅ and M are as defined above and Z represents R₆NH— wherein R₆ represents ethyl substituted at the 2-position by R₉ wherein R₉ represents cyano, nitro, —S(O)_{q}R₁₀, —C(O)alkyl, alkoxycarbonyl, alkylarninocarbonyl, dialkylaminocarbonyl, aminosulfonyl, alkylaminosulfonyl or dialkylaminosulfonyl, may be prepared by the reaction of the corresponding compound of formula (I) wherein Z is amino with a compound of formula (III):

R₉—CH═CH₂ (III)

wherein R₉ is defined above. The reaction may be performed preferably in the presence of a base such as sodium hydride, an alkali metal hydroxide for example potassium hydroxide, or a tetraalkylammonium hydroxide for example N-benzyltrimethylammonium hydroxide in a solvent such as N,N-dimethylformamide, tetrahydrofuran, toluene, ethanol or water, and at a temperature from −20° C. to the reflux temperature.

According to a further feature of the invention compounds of general formula (I) wherein R₁, R₂, R₃, R₄, R₅ and M are as defined above and Z represents —NHR₆ or —NR₇R₈ wherein R₇ and R₈ represent alkyl or haloalkyl optionally substituted by one or more R₉, may be prepared by the reaction of the corresponding compound of formula (I) wherein Z is amino with a compound of formula (IV):

R₁₁—Y (IV)

wherein R₁₁ is alkyl or haloalkyl optionally substituted by one or more R₉ and Y represents a leaving group, preferably halogen (for example chlorine). The reaction may be performed in the presence of a base such as potassium hydroxide, potassium methoxide, sodium hydride or triethylamine in an inert solvent such as N,N-dimethylformamide, tetrahydrofuran, toluene or ether, and at a temperature from −20° C. to the reflux temperature.

According to a further feature of the present invention compounds of general formula (1) wherein Z represents alkoxycarbonyl may be prepared by the reaction of the corresponding compound of formula (1) in which Z is carboxy with an alcohol of formula (V):

R₁₂OH (V)

wherein R₁₂ is alkyl. The above reaction is preferably performed in the presence of an acid catalyst such as sulphuric acid generally in the presence of excess of the alcohol or optionally in a co-solvent at a temperature from 0° C. to the reflux temperature. Alternatively the reaction may be performed using a coupling reagent such as dicyclohexylcarbodiimide (DCC) in an inert solvent.

According to a further feature of the present invention compounds of general formula (I) wherein Z represents alkylthiocarbonyl may be prepared by the reaction of the corresponding compound of formula (1) in which Z is carboxy with an thiol of formula (VI):

R₁₂SH (VI)

wherein R₁₂ is alkyl. The reaction may be performed using a coupling reagent such as dicyclohexylcarbodiimide (DCC) in an inert solvent.

According to a further feature of the present invention compounds of general formula (I) wherein Z represents R₁₃CH(Cl)CH₂— wherein R₁₃ represents alkyl may be prepared by the diazotisation of the corresponding compound of formula (I) in which Z represents amino followed by reaction with a compound of formula (VII):

R₁₃CH═CH₂ (VII)

The reaction is generally carried out using an alkyl nitrite such as tert-butyl nitrite in the presence of a copper salt such as copper (II) chloride in a solvent such as acetonitrile at a temperature from −10° C. to 50C.

According to a further feature of the present invention compounds of general formula (I) wherein Z represents —C(O)alkyl may be prepared by the oxidation of the corresponding compound of formula (VIII):

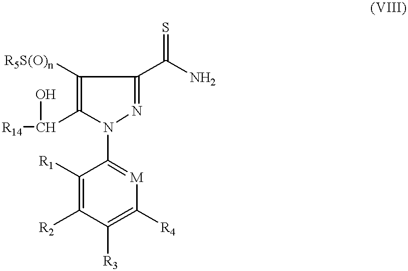

wherein R₁, R₂, R₃, R₄, R₅ and M are as defined above and R₁₄ represents alkyl. The reaction may be performed using for example a mixture of chromic acid and sulphuric acid in a solvent such as water and acetone at a temperature from 0° C. to 60° C. Compounds of formula (VIII) are novel and thus constitute a further feature of the present invention.

According to a further feature of the invention compounds of formula (I) in which Z represents R₁₃CH═CH— may be prepared by the dehydrochlorination of the corresponding compound of formula (I) in which Z represents R₁₃CH(Cl)CH₂—. The reaction is generally performed using a base such as sodium hydroxide or triethylamine in an inert solvent for example dichloromethane or tetrahydrofuran at a temperature from −70° C. to the reflux temperature.

According to a further feature of the present invention compounds of formula (I) in which the substituent Z is hydrazino, 1H-pyrrol-1-yl or 1H-pyrazol-1-yl may be prepared according to the procedures described in EP 0352944.

Intermediates of general formula (I) wherein Z represents carboxy may be prepared by the oxidation of the corresponding compound of formula (I) in which Z represents formyl. The reaction is generally carried out using potassium permanganate or chromic acid in a solvent such as water at a temperature of from 0° C. to the reflux temperature.

Intermediates of formula (VIII) can be prepared by reaction of the corresponding compounds of formula (I) in which Z represents a formyl group with an organometallic reagent of formula R₁₄Q wherein Q is preferably an alkali or alkaline earth metal for example lithium or a magnesium halide (Grignard) reagent. The reaction may be performed in an inert solvent such as ether or tetrahydrofuran and at a temperature from −78° C. to the reflux temperature of the solvent.

Intermediates of formula (I) in which Z represents a formyl group may be prepared by the oxidation of the corresponding compound of formula (I) in which Z represents R₁₃CH═CH—. The reaction is generally performed using a reagent such as ozone or sodium metaperiodite in an inert solvent for example dichloromethane at a temperature from −100° C. to 100° C.

Certain compounds of formula (II) are novel and as such form a further feature of the present invention.

Intermediates of formula (III) and (IV) are known or may be prepared by known methods.

Illustrative specific compounds of formula (I) of the invention are set forth in Table 1 below. In the Table c-Pr means cyclopropyl and CH₂C₂H means a propargyl group.

TABLE 1

| Cmpd | | | | | | | |
|---|---|---|---|---|---|---|---|
| No. | R₅ S(O)ₙ | Z | R₁ | R₂ | R₃ | R₄ | M |
| 1 | SCH₃ | NH₂ | Cl | H | CF₃ | H | CCl |
| 2 | SCF₃ | NH₂ | Cl | H | CF₃ | H | CCl |
| 3 | SO₂CFCl₂ | H | Cl | H | Cl | H | CCl |
| 4 | SOCH₃ | NH₂ | Cl | H | CF₃ | H | CCl |
| 5 | SO₂CH₃ | NH₂ | Cl | H | CF₃ | H | CCl |
| 6 | SOCH₃ | NH₂ | Br | H | CF₃ | H | CCl |
| 7 | SOCH₃ | NH₂ | Cl | H | OCF₃ | H | CCl |
| 8 | SOC₂H₅ | NH₂ | Cl | H | CF₃ | H | CCl |
| 9 | SOCH₃ | NH₂ | H | H | CF₃ | H | CCl |
| 10 | SOC₂H₅ | NH₂ | Br | H | CF₃ | H | CCl |
| 11 | SOC₂H₅ | NH₂ | Cl | H | OCF₃ | H | CCl |
| 12 | SC₂H₅ | NH₂ | Cl | H | CF₃ | H | CCl |
| 13 | SCH₂CH═CH₂ | NH₂ | Br | H | CF₃ | H | CCl |
| 14 | SOC₂H₅ | NHCOCH₃ | Cl | H | CF₃ | H | CF |
| 15 | SO₂CH₃ | NH₂ | Cl | H | CF₃O | H | CCl |
| 16 | SOCH₃ | NHC₂H₅ | Cl | H | CF₃ | H | CCl |
| 17 | SOC₂H₅ | NHCH₃ | F | H | CF₃ | CH₃ | CCl |
| 18 | SO-c-Pr1 | NHCH₂OCH₃ | Br | H | CF₃ | H | CCl |
| 19 | SOCH₃ | NHCH₂CONH₂ | Cl | H | CF₃ | H | N |
| 20 | S-i-C₃H₇ | NH₂ | Cl | H | CF₃O | H | CCl |
| 21 | SO₂C₂H₅ | NHCHO | Cl | CH₃ | CF₃S | H | CBr |
| 22 | SCH₃ | NH₂ | Cl | H | CF₃ | CH₃ | CF |
| 23 | SOCH₃ | NHSO₂C₂H₅ | Cl | H | CHF₂ | H | CCl |
| 24 | SO-c-Pr2 | Br | F | H | CClF₂ | H | CF |
| 25 | SCH₂C₂H | H | Cl | H | CF₃ | H | CBr |
| 26 | SOC₂H₅ | CH₃ | Cl | H | CF₃O | H | CCl |
| 27 | SCH₂CH═CH₂ | NHNH₂ | Br | CH₃ | CF₃ | H | CCl |
| 28 | SOCH₃ | NH₂ | Cl | H | CF₃ | H | CCH₃ |
| 29 | SCH₃ | NHSC₂H₅ | Cl | H | CF₃ | H | CCl |
| 30 | SOCH₃ | NHCH₂CH₂CN | Cl | H | CF₃ | H | CCl |
| 31 | SO₂CH₃ | NHCH₂CH₂OCH₃ | Cl | H | CF₃ | CH₃ | CCl |
| 32 | SO-i-C₃H₇ | NH₂ | Cl | H | CF₃ | H | N |
| 33 | SOC₂H₅ | COCH₃ | Br | H | CF₃O | H | CCl |
| 34 | SOCH₃ | NHCH₃ | Cl | H | CF₃ | H | CCl |
| 35 | SOCH₃ | NH₂ | Br | H | CF₃ | H | N |
| 36 | SOC₂H₅ | NH(CH₂)₂CONH₂ | Cl | H | CF₃ | H | CCl |
| 37 | SOCH₃ | CH═CH₂ | F | CH₃ | CF₃ | H | CF |
| 38 | SO₂CH₃ | NH₂ | Cl | H | CF₃ | H | CNO₂ |
| 39 | SO-c-Pr3 | NHCO₂CH₃ | Cl | H | CF₂Cl | H | CCl |
| 40 | SCH₃ | NHSCCl₃ | Cl | H | CFCl₂ | H | CCl |
| 41 | SCH₂C₂H | NHCH₂CH₂SCH₃ | F | H | CF₃ | H | CCl |
| 42 | SOC₂H₅ | NHCH₂CH₂SOC₂H₅ | Cl | H | CF₃ | H | CCl |
| 43 | SOC₂H₅ | CH₃ | H | H | CF₃ | H | CCl |
| 44 | SOC₂H₅ | N(CH₃)₂ | H | H | CF₃ | H | CCl |
| 45 | SOCH₃ | N(CH₃)₂ | Cl | H | CF₃ | H | CBr |

The invention is illustrated by the following examples, which are not considered as limiting the invention but are given to better enable use of it.

### EXAMPLE 1

5-Amino-1-(2,6-dichloro-4-trifluoromethylphenyl)-4-methylthiopyrazole-3-thiocarboxamide

To a stirred solution of 5-amino-1-(2,6-dichloro-4-trifluoromethylphenyl)-3-cyano-4-methylthiopyrazole (4.7 g) and 5 drops of 15-crown-5 in degassed N,N-dimethylformamide was added, over a few minutes, a solution of sodium hydrosulfide hydrate (0.79 g) in degassed water. The mixture was stirred at 20° C. for 18 hours, evaporated and the residue purified by flash chromatography on silica gel, eluting with 2:1 hexane/acetone to give 5-amino-1-(2,6-dichloro-4-trifluoromethylphenyl)-4-methylthiopyrazole-3-thiocarboxamide (0.38 g), m.p.161° C. Compound 1.

In a manner similar to that employed above, Compound numbers 2 to 11 of general formula (I) shown in Table 2 below were also prepared.

TABLE 2

| Cmpd No. | R₁ | R₂ | R₃ | R₄ | M | R₅ S(O)ₙ | Z | m.p.° C. |
|---|---|---|---|---|---|---|---|---|
| 2 | Cl | H | CF₃ | H | CCl | SCF₃ | NH₂ | 182 |
| 3 | Cl | H | Cl | H | CCl | SO₂CFCl₂ | H | 180.5 |
| 4 | Cl | H | CF₃ | H | CCl | SOCH₃ | NH₂ | 192 |
| 5 | Cl | H | CF₃ | H | CCl | SO₂CH₃ | NH₂ | 231 |
| 6 | Br | H | CF₃ | H | CCl | SOCH₃ | NH₂ | 188.5 |
| 7 | Cl | H | OCF₃ | H | CCl | SOCH₃ | NH₂ | 137 |
| 8 | Cl | H | CF₃ | H | CCl | SOC₂H₅ | NH₂ | 167 |
| 9 | H | H | CF₃ | H | CCl | SOCH₃ | NH₂ | 153.5 |
| 10 | Br | H | CF₃ | H | CCl | SOC₂H₅ | NH₂ | 113.5 |
| 11 | Cl | H | OCF₃ | H | CCl | SOC₂H₅ | NH₂ | 102 |

### REFERENCE EXAMPLE 1

5-Amino-1-(2,4,6-trichlorophenyl)-3-cyanopyrazole

Sodium nitrite (27.6 g) was added slowly to concentrated sulphuric acid (106 ml) and the mixture allowed to cool to 30° C. To the stirred mixture was added acetic acid (216 ml) over 30 minutes, which was cooled to 200 C and a solution of 2,4,6-trichloroaniline (70.7 g) in acetic acid added over 30-minutes, maintaining at 20-25° C. The mixture was heated at 520 C for 30 minutes, then fed over 20 minutes, to a solution of ethyl 2,3-dicyanopropionate (52.0 g) in 36% aqueous acetic acid (774 ml), while cooling to 10-15° C. The reaction mixture was allowed to warm to 20° C., diluted (water) and extracted (dichloromethane). The organic layer was then washed with dilute ammonium hydroxide solution and stirred with 29% ammonium hydroxide solution for a 17-hours. The organic phase was washed with water then with hydrochloric acid (1N), dried (MgSO₄) and evaporated. Crystallization from toluene afforded 5-amino-1-(2,4,6-trichlorophenyl)-3-cyanopyrazole (32.82 g), ¹H NMR (CDC13): NH₂ (2H, br s, 3.84 ppm), 4-H (1H, s, 5.97 ppm), arom.(2H, s, 7.53 ppm).

### REFERENCE EXAMPLE 2

5-Amino-4-dichlorofluoromethylthio-1-(2,4,6-trichlorophenyl-3-cyanopyrazole

To a stirred solution of 5-amino-1-(2,4,6-trichlorophenyl)-3-cyanopyrazole (8.0 g, 0.028 mol) in acetic acid (70 ml) was fed dichlorofluoromethylsulfenyl chloride (4.4 ml) over 5-minutes. The mixture was allowed to stir at 20° C. for 65-hours, then poured into water and extracted (dichloromethane). The organic phase was washed with cold sodium bicarbonate solution then water, dried (MgSO₄) and evaporated. Column chromatography on silica gel eluting with 3:1 dichloromethane/hexane, afforded 5-amino-4-dichlorofluoromethylthio-1-(2,4,6-trichlorophenyl)-3-cyanopyrazole (6.06 g), m.p.189-191° C.

### REFERENCE EXAMPLE 3

4-Dichlorofluoromethvlthio-1-(2,4,6-trichlorophenyl)-3-cyanopyrazole

A solution of 5-amino-4-dichlorofluoromethylthio-1-(2,4,6-trichlorophenyl)-3-cyanopyrazole (2.19 g)and tert-butyl nitrite (90%, 4.1 ml) in tetrahydrofuran was heated under reflux for 3.5 hours and evaporated. The residue was purified by flash-chromatography on silica gel, eluting with dichloromethane to give 4-dichlorofluoromethylthio-1-(2,4,6-trichlorophenyl)-3-cyanopyrazole (1.56 g), m.p.106-108° C.

### REFERENCE EXAMPLE 4

4-Dichlorofluoromethylsulfonyl-3-cyano-1-(2,4,6-trichlorophenyl)pyrazole

A solution of 4-dichlorofluoromethylthio-3-cyano-1-(2,4,6-trichlorophenyl)pyrazole (0.80 g) and 3-chloroperbenzoic acid (80%, 0.86 g) in chloroform was heated under reflux for 2 hours, cooled, diluted (ethyl acetate) and washed in sequence with 10% sodium hydrogen sulfite, 2N NaOH, water and brine. The dried (MgSO₄) solution was evaporated and purified by flash-chromatography on silica gel eluting with dichloromethane to give 4-dichlorofluoromethylsulfonyl-1-(2,4,6-trichlorophenyl)-3-cyanopyrazole (0.41 g), m.p.143.5-145° C.

### REFERENCE EXAMPLE 5

5-Amino-1-(2-chloro-4-trifluoromethylphenyl)-3-cyano-4-methylsulfinylpyrazole

Step 1

Bromine (0.5 ml) was added over 10 minutes to a stirred solution of sodium thiocyanate (1.7 g) in anhydrous methanol at −65° C. A solution of 5-amino-1-(2-chloro-4-trifluoromethylphenyl)-3-cyanopyrazole (1.5 g) in anhydrous methanol was added over 10-minutes and the stirred mixture allowed to warm to 20° C. over 16-hours. After pouring into water the precipitate was collected and dried to give 5-amino-1-(2-chloro-4-trifluoromethylphenyl)-3-cyano-4-thiocyanatopyrazole (1.64 g). HPLC (C-18 column, eluting with 3:1 CH₃CN/H₂O at 1.0 ml/min.) showed the compound as a peak of 86.6% area at 5.11 minutes.

Step 2

Iodomethane (0.7 ml) was injected into a stirred suspension of 5-amino-1(2-chloro-4-trifluoromethylphenyl)-3-cyano-4-thiocyanatopyrazole (1.64 g), in methanol at 40 C. A 10% aqueous solution of sodium hydroxide (2.8 ml) was added and the reaction mixture stirred for 1 hour at 40 C , poured into water and extracted with dichloromethane and ethyl acetate. The dried (Na2SO4) combined organic phase was evaporated and purified by flash-chromatography on silica gel eluting with 4:1 hexane/ethyl acetate to give, after trituration with hexane/dichloromethane, 5-amino-1-(2-chloro-4-trifluoromethylphenyl)-3-cyano-4-methylthiopyrazole (0.4 g), m.p.129-132° C.

Step 3

To a stirred solution of 5-amino-1-(2-chloro-4-trifluoromethylphenyl)-3-cyano-4-methylthiopyrazole (3.0 g) in trifluoroacetic acid (20 ml) was added 30% hydrogen peroxide (0.5 ml) with cooling at 40 C. The mixture was brought to 20° C. over 2 hours, then poured onto ice and the solid water-washed and dried. Flash-column chromatography on silica gel gave 5-amino-1-(2-chloro-4-trifluoromethylphenyl)-3-cyano-4-methylsulfinylpyrazole (0.69 g) m.p.146-147° C.

### REFERENCE EXAMPLE 6

In a manner similar to that employed in Reference Example 5, Steps 1 and 2, the following compounds were also prepared: 5-amino-1-(2,6-dichloro-4-trifluoromethoxyphenyl)-3-cyano-4-methylthiopyrazole, m.p. 147-148° C., and 5-amino-1-(2-bromo-6-chloro-4-trifluoromethylphenyl)-3-cyano-4-methylthiopyrazole, m.p. 178-180° C.

### REFERENCE EXAMPLE 7

In a manner similar to that employed in Reference Example 5, Step 3, the following compounds were also prepared:

5-amino-1-(2,6-dichloro-4-trifluoromethoxyphenyl)-3-cyano-4-methylsulfinylpyrazole, m.p. 137-138° C., and

5-amino-1-(2-bromo-6-chloro-4-trifluoromethylphenyl)-3-cyano-4methylsulfinylpyrazole, m.p. 150-151° C.

Biological Efficacy

The following methods were used to apply the compounds of the invention and to observe the results obtained therewith: a foliar/contact spray on sucking (aphids) or chewing (Lepidoptera) insects.

The species tested were as follows:

| GENUS, SPECIES | COMMON NAME | ABBREVIATION |
|---|---|---|
| Aphis gossypii | cotton leaf aphid | APHIGO |
| Schizaphis graminum | greenbug | TOXOGR |
| Musca domestica | housefly | MUSCDO |

The Soil Drench Test (Systemic Activity)

Cotton and sorghum plants were established in pots. One day prior to treatment, each pot was infested with about 25 aphids of a mixed population. Cotton plants were infested with aphids and sorghum plants were infested with the greenbug. The selected compound of formula (I) was applied to the soil surface in a dilution that delivered the equivalent of 10.0 ppm soil concentration by weight. Aphid counts were obtained at 5 DAT (=days after treatment). The number of aphids on the treated plants was compared to the number of those on the untreated control plants.

The Housefly Bait/Contact Test (Musca domestica)

About 25 four to six-day-old adult houseflies were anesthetized and placed in a cage with a sugar water bait solution containing the compound. The concentration of the selected compound of formula (I) in the bait solution was 100 ppm. After 24 hours, flies which showed no movement on stimulation were considered dead.

Foliar/Contact Test with A. gossypii

Aphid-infested cotton plants were placed on a revolving turntable, and sprayed to runoff with a 100 ppm formulation of the selected compound of formula (I). The treated, A. gossypii-infested plants were held for three days after treatment, after which the dead aphids were counted.

Compounds 1-11 of the invention showed activity against Aphis gossypii and Schizaphis graminum at 10 ppm in the above systemic test; against Musca domestica at 100 ppm in the above bait/contact test; and against Aphis gossypii at 100 ppm in the above foliar/contact test.

The present invention provides a method for the systemic control of arthropods at a locus, especially some insects or mites which feed on the above ground portions of plants. Control of such foliar pests may be provided by direct foliar application or by application by for example soil spray or granule application to the plant roots or plant seeds with subsequent systemic translocation to the above ground portions of the plants. Such systemic activity includes the control of insects which reside not only at the point of application but at a remote part of the plant for example by translocation from one side of a leaf to the other or from a treated leaf to an untreated leaf. Examples of the classes of insect pests which may be systemically controlled by the compounds of the invention include the Homoptera order (piercing-sucking), Hemiptera order (piercing-sucking), and Th sanoptera order. The invention is especially appropriate for aphids and thrips.

As is evident from the foregoing pesticidal uses, the present invention provides pesticidally active compounds and methods of use of said compounds for the control of a number of pest species which includes: arthropods, especially insects or mites; plant nematodes; or helminth or protozoan pests. The compounds of formula (I) or pesticidally acceptable salts thereof thus are advantageously employed in practical uses, for example, in agricultural or horticultural crops, forestry, veterinary medicine or livestock husbandry, or in public health. From this point forward, whenever the term “compounds of formula (I)” is used this term embraces compounds of formula (I) and their pesticidally acceptable salts. The term “compound of formula (I)” embraces a compound of formula (I) and a pesticidally acceptable salt thereof.

The present invention therefore provides a method of control of pests at a locus which comprises the treatment of the locus (e.g., by application or administration) with an effective amount of a compound of formula (I) or a pesticidally acceptable salt thereof, wherein the substituent groups are as hereinbefore defined. The locus includes, for example, the pest itself or the place (plant, animal, field, structure, premises, forest, orchard, waterway, soil, plant or animal product, or the like) where the pest resides or feeds.

The compounds of this invention may in addition be used to control soil insects, such as corn rootworm, termites (especially for protection of structures), root maggots, wireworms, root weevils, stalkborers, cutworms, root aphids, or grubs. They may also be used to provide activity against plant pathogenic nematodes, such as root-knot, cyst, dagger, lesion, or stem or bulb nematodes, or against mites. For the control of soil pests, for example corn rootworm, the compounds are advantageously applied to or incorporated at an effective rate into the soil in which crops are planted or to be planted or to the seeds or growing plant roots.

In the area of public health, the compounds are especially useful in the control of many insects, especially filth flies or other Dipteran pests, such as houseflies, stableflies, soldierflies, hornflies, deerflies, horseflies, midges, punkies, blackflies, or mosquitoes.

Compounds of the invention may be used in the following applications and on the following pests including arthropods, especially insects or mites, nematodes, or helminth or protozoan pests:

In the protection of stored products, for example cereals, including grain or flour, groundnuts, animal feedstuffs, timber or household goods, e.g. carpets and textiles, compounds of the invention are useful against attack by arthropods, more especially beetles, including weevils, moths or mites, for example Ephestia spp. (flour moths), Anthrenus spp. (carpet beetles), Tribolium spp. (flour beetles), Sitophilus spp. (grain weevils) or Acarus spp. (mites).

In the control of cockroaches, ants or termites or similar arthropod pests in infested domestic or industrial premises or in the control of mosquito larvae in waterways, wells, reservoirs or other running or standing water.

For the treatment of foundations, structures or soil in the prevention of the attack on building by termites, for example, Reticulitermes spp., Heterotermes spp., Coptotermes spp.

In agriculture against adults, larvae and eggs of Lepidoptera (butterflies and moths), e.g. Heliothis spp. such as Heliothis virescens (tobacco budworm), Heliothis armigera and Heliothis zea. Against adults and larvae of Coleoptera (beetles) e.g. Anthonomus spp. e.g. grandis (cotton boll weevil), Leptinotarsa decemlineata (Colorado potato beetle), Diabrotica spp. (corn rootworms). Against Heteroptera (Hemiptera and Homoptera) e.g. Psylla spp., Bemisia spp., Trialeurodes spp., A his spp., Myzus spp., Megoura viciae, Ph lloxera spp., Nephotettix spp. (rice leaf hoppers), Nilaparvata spp. Against Diptera e.g. Musca spp. Against Thysanoptera such as Thrips tabaci. Against Orthoptera such as Locusta and Schistocerca spp., (locusts and crickets) e.g. Gryllus spp., and Acheta spp. for example, Blatta orientalis, Periplaneta americana, Blatella germanica Locusta migratoria migratorioides and Schistocerca gregaria. Against Collembola e.g. Periplaneta spp. and Blattela spp. (roaches). Against Isoptera e.g. Coptotermes spp. (termites).

Against arthropods of agricultural significance such as Acari (mites) e.g. Tetranvchus spp., and Panonychus spp.

Against nematodes which attack plants or trees of importance to agriculture, forestry or horticulture either directly or by spreading bacterial, viral, mycoplasma or fungal diseases of the plants. For example root-knot nematodes such as Meloidogyne spp. (e.g. M. incognita).

In the field of veterinary medicine or livestock husbandry or in the maintenance of public health against arthropods, helminths or protozoa which are parasitic internally or externally upon vertebrates, particularly warm-blooded vertebrates, for example domestic animals, e.g. cattle, sheep, goats, equines, swine, poultry, dogs or cats, for example Acarina, including ticks (e.g. Ixodes spp., Boophilus spp. e.g. Boophilus microplus, Rhipicephalus spp. e.g. Rhipicephalus appendiculatusOrnithodorus spp. (e.g. Ornithodorus moubata) and mites (e.g. Damalinia spp.); Diptera (e.g. Aedes spp., Anopheles spp., Musca spp., Hypoderma spp.); Hemiptera.; Dictyoptera (e.g. Periplaneta spp., Blatella spp.); Hymenoptera; for example against infections of the gastro-intestinal tract caused by parasitic nematode worms, for example members of the family Trichostrongylidae; in the control and treatment of protozoal diseases caused by, for example, Eimeria spp. e.g. Trypanosoms cruzi, Leishaminia spp., Plasmodium spp., Babesis spp., Trichomonadidae spp., Toxoplasma spp. and Theileria spp.

In practical use for the control of arthropods, especially insects or mites, or nematode pests of plants, a method, for example, comprises applying to the plants or to the medium in which they grow an effective amount of a compound of the invention. For such a method, the active compound is generally applied to the locus in which the arthropod or nematode infestation is to be controlled at an effective rate in the range of about 5 g to about 1 kg of the active compound per hectare of locus treated. Under ideal conditions, depending on the pest to be controlled, a lower rate may offer adequate protection. On the other hand, adverse weather conditions, resistance of the pest or other factors may require that the active ingredient be used at higher rates. The optimum rate depends usually upon a number of factors, for example, the type of pest being controlled, the type or the growth stage of the infested plant, the row spacing or also the method of application. More preferably an effective rate range of the active compound is from about 50g/ha to about 400 g/ha.

When a pest is soil-borne, the active compound generally in a formulated composition, is distributed evenly over the area to be treated (ie, for example broadcast or band treatment) in any convenient manner and is applied at rates from about 5 g to about lkg ai/ha, preferably from about 50 to about 250 g ai/ha. When applied as a root dip to seedlings or drip irrigation to plants the liquid solution or suspension contains from about 0.075 to about 1000 mg ai/l, preferably from about 25 to about 200 mg ai/l. Application may be made, if desired, to the field or crop-growing area generally or in close proximity to the seed or plant to be protected from attack. The active component can be washed into the soil by spraying with water over the area or can be left to the natural action of rainfall. During or after application, the formulated compound can, if desired, be distributed mechanically in the soil, for example by ploughing, disking, or use of drag chains. Application can be prior to planting, at planting, after planting but before sprouting has taken place, or after sprouting.

The compounds of the invention and methods of control of pests therewith are of particular value in the protection of field, forage, plantation, glasshouse, orchard or vineyard crops, of ornamentals, or of plantation or forest trees, for example: cereals (such as wheat or rice), cotton, vegetables (such as peppers), field crops (such as sugar beets, soybeans or oil seed rape), grassland or forage crops (such as maize or sorghum), orchards or groves (such as of stone or pit fruit or citrus), ornamental plants, flowers or vegetables or shrubs under glass or in gardens or parks, or forest trees (both deciduous and evergreen) in forests, plantations or nurseries.

They are also valuable in the protection of timber (standing, felled, converted, stored or structural) from attack, for example, by sawflies or beetles or termites.

They have applications in the protection of stored products such as grains, fruits, nuts, spices or tobacco, whether whole, milled or compounded into products, from moth, beetle, mite or grain weevil attack. Also protected are stored animal products such as skins, hair, wool or feathers in natural or converted form (e.g. as carpets or textiles) from moth or beetle attack as well as stored meat, fish or grains from beetle, mite or fly attack. Additionally, the compounds of the invention and methods of use thereof are of particular value in the control of arthropods, helminths or protozoa which are injurious to, or spread or act as vectors of diseases domestic animals, for example those hereinbefore mentioned, and more especially in the control of ticks, mites, lice, fleas, midges, or biting, nuisance or myiasis flies. The compounds of the invention are particularly useful in controlling arthropods, helminths or protozoa which are present inside domestic host animals or which feed in or on the skin or suck the blood of the animal, for which purpose they may be administered orally, parenterally, percutaneously or topically.

Furthermore, compounds of the invention may be useful for coccidiosis, a disease caused by infections from protozoan parasites of the genus Eimeria. It is an important potential cause of economic loss in domestic animals and birds, particularly those raised or kept under intensive conditions. For example, cattle, sheep, pigs or rabbits may be affected, but the disease is especially important in poultry, particularly in chickens. Administration of a small amount of a compound of the invention, preferably by a combination with feed is effective in preventing or greatly reducing the incidence of coccidiosis. The compounds are effective against both the cecal form and the intestinal forms. Furthermore, the compounds of the invention may also exert an inhibiting effect on oocytes by greatly reducing the number and sporulation of those produced. The poultry disease is generally spread by the birds picking up the infectious organism in droppings in or on contaminated litter, ground, food, or drinking water. The disease is manifested by hemorrhage, accumulation of blood in the ceca, passage of blood to the droppings, weakness and digestive disturbances. The disease often terminates in the death of the animal, but the fowl which survive severe infections have had their market value subtantially reduced as a result of the infection.

The compositions hereinafter described for application to growing crops or crop growing loci or as a seed dressing may, in general, alternatively be employed for topical application to animals or in the protection of stored products, household goods, property or areas of the general environment. Suitable means of applying the compounds of the invention include:

to growing crops as foliar sprays, dusts, granules, fogs or foams or also as suspensions of finely divided or encapsulated compositions as soil or root treatments by liquid drenches, dusts, granules, smokes or foams; to seeds of crops via application as seed dressings by liquid slurries or dusts;

to animals infested by or exposed to infestation by arthropods, helminths or protozoa, by parenteral, oral or topical application of compositions in which the active ingredient exhibits an immediate and/or prolonged action over a period of time against the arthropods, helminths or protozoa, for example by incorporation in feed or suitable orally-ingestible pharmaceutical formulations, edible baits, salt licks, dietary supplements, pour-on formulations, sprays, baths, dips, showers, jets, dusts, greases, shampoos, creams, wax smears or livestock self-treatment systems;

to the environment in general or to specific locations where pests may lurk, including stored products, timber, household goods, or domestic or industrial premises, as sprays, fogs, dusts, smokes, wax-smears, lacquers, granules or baits, or in tricklefeeds to waterways, wells, reservoirs or other running or standing water;

to domestic animals in feed to control fly larvae feeding in their feces;

In practice, the compounds of the invention most frequently form parts of compositions. These compositions can be employed to control: arthopods, especially insects or mites; nematodes; or helminth or protozoan pests. The compositions may be of any type known in the art suitable for application to the desired pest in any premises or indoor or outdoor area or by internal or external administration to vertebrates. These compositions contain at least one compound of formula (I) or a pesticidally acceptable salt thereof, such as described earlier, as the active ingredient in combination or association with one or more other compatible components which are for example, solid or liquid carriers or diluents, adjuvants, surface-active-agents, or the like appropriate for the intended use and which are agronomically or medicinally acceptable. These compositions, which may be prepared by any manner known in the art, likewise form a part of this invention.

These compositions may also contain other kinds of ingredients such as protective colloids, adhesives, thickeners, thixotropic agents, penetrating agents, spray oils (especially for acaridical use), stabilizers, preservative agents (especially mold preservatives), sequestering agents, or the like, as well as other known active ingredients with pesticidal properties (particularly insecticidal, miticidal, nematicidal, or fungicidal) or with properties regulating the growth of plants. More generally, the compounds employed in the invention may be combined with all the solid or liquid additives corresponding to the usual techniques of formulation.

Compositions, suitable for applications in agriculture, horticulture, or the like include formulations suitable for use as, for example, sprays, dusts, granules, fogs, foams, emulsions, or the like.

The effective use doses of the compounds employed in the invention can vary within wide limits, particularly depending on the nature of the pest to be eliminated or degree of infestation, for example, of crops with these pests. In general, the compositions according to the invention usually contain about 0.05 to about 95% (by weight) of one or more active ingredients according to the invention, about 1 to about 95% of one or more solid or liquid carriers and, optionally, about 0.1 to about 50% of one or more other compatible components, such as surface-active agents or the like.

In the present account, the term “carrier” denotes an organic or inorganic ingredient, natural or synthetic, with which the active ingredient is combined to facilitate its application, for example, to the plant, to seeds or to the soil. This carrier is therefore generally inert and it must be acceptable (for example, agronomically acceptable, particularly to the treated plant).

The carrier may be a solid, for example, clays, natural or synthetic silicates, silica, resins, waxes, solid fertilizers (for example ammonium salts), ground natural minerals, such as kaolins, clays, talc, chalk, quartz, attapulgite, montmorillonite, bentonite or diatomaceous earth, or ground synthetic minerals, such as silica, alumina, or silicates especially aluminium or magnesium silicates. As solid carriers for granules the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

The carrier may also be liquid, for example: water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulphoxide, or N-methylpyrrolidone; liquefied gases; or the like or a mixture thereof.

The surface-active agent may be an emulsifying agent, dispersing agent or wetting agent of the ionic or non-ionic type or a mixture of such surface-active agents. Amongst these are e.g., salts of polyacrylic acids, salts of lignosulphonic acids, salts of phenolsulphonic or naphthalenesulphonic acids, polycondensates of ethylene oxide with fatty alcohols or fatty acids or fatty esters or fatty amines, substituted phenols (particularly alkylphenols or arylphenols), salts of sulphosuccinic acid esters, taurine derivatives (particularly alkyltaurates), phosphoric esters of alcohols or of polycondensates of ethylene oxide with phenols, esters of fatty acids with polyols, or sulphate, sulphonate or phosphate functional derivatives of the above compounds. The presence of at least one surface-active agent is generally essential when the active ingredient and/or the inert carrier are only slightly water soluble or are not water soluble and the carrier agent of the composition for application is water.

Compositions of the invention may further contain other additives such as adhesives or colorants. Adhesives such as carboxymethylcellulose or natural or synthetic polymers in the form of powders, granules or lattices, such as arabic gum, polyvinyl alcohol or polyvinyl acetate, natural phospholipids, such as cephalins or lecithins, or synthetic phospholipids can be used in the formulations. It is possible to use colorants such as inorganic pigments, for example: iron oxides, titanium oxides or Prussian Blue; organic dyestuffs, such as alizarin dyestuffs, azo dyestuffs or metal phthalocyanine dyestuffs; or trace nutrients such as salts of iron, manganese, boron, copper, cobalt, molybdenum or zinc.

Compositions containing compounds of formula (I), or pesticidally acceptable salts thereof, which may be applied to control arthropod, plant nematode, helminth or protozoan pests, may also contain synergists (e.g. piperonyl butoxide or sesamex), stabilizing substances, other insecticides, acaricides, plant nematocides, anthelmintics or anticoccidials, fungicides (agricultural or veterinary as appropriate, e.g. benomyl and iprodione), bactericides, arthropod or vertebrate attractants or repellents or pheromones, deodorants, flavouring agents, dyes, or auxiliary therapeutic agents, e.g. trace elements. These may be designed to improve potency, persistence, safety, uptake where desired, spectrum of pests controlled or to enable the composition to perform other useful functions in the same animal or area treated.

Examples of other pesticidally-active compounds which may be included in, or used in conjunction with the compositions of the present invention are: acephate, chlorpyrifos, demeton-S-methyl, disulfoton, ethoprofos, fenitrothion, fenamiphos, fonofos, isazophos, isofenphos, malathion, monocrotophos, parathion, phorate, phosalone, pirimiphos-methyl, terbufos, triazophos, cyfluthrin, cypermethrin, deltamethrin, fenpropathrin, fenvalerate, permethrin, tefluthrin, aldicarb, carbosulfan, methomyl, oxamyl, pirimicarb, bendiocarb, teflubenzuron, dicofol, endosulfan, lindane, benzoximate, cartap, cyhexatin, tetradifon, avermectins, ivermectins, milbemycins, thiophanate, trichlorfon, dichlorvos, diaveridine or dimetriadazole.

For their agricultural application, the compounds of the formula (I), or pesticidally acceptable salts thereof, are therefore generally in the form of compositions, which are in various solid or liquid forms.

Solid forms of compositions which can be used are dusting powders (with a content of the compound of formula (I), or a pesticidally acceptable salt thereof, ranging up to 80%), wettable powders or granules (including water dispersible granules), particularly those obtained by extrusion, compacting, impregnation of a granular carrier, or granulation starting from a powder (the content of the compound of formula (I), or a pesticidally acceptable salt thereof, in these wettable powders or granules being between about 0.5 and about 80%). Solid homogenous or heterogenous compositions containing one or more compounds of formula (I), or pesticidally acceptable salts thereof, for example granules, pellets, briquettes or capsules, may be used to treat standing or running water over a period of time. A similar effect may be achieved using trickle or intermittent feeds of water dispersible concentrates as described herein.

Liquid compositions, for example, include aqueous or non-aqueous solutions or suspensions (such as emulsifiable concentrates, emulsions, flowables, dispersions, or solutions) or aerosols. Liquid compositions also include, in particular, emulsifiable concentrates, dispersions, emulsions, flowables, aerosols, wettable powders (or powder for spraying), dry flowables or pastes as forms of compositions which are liquid or intended to form liquid compositions when applied, for example as aqueous sprays (including low and ultra-low volume) or as fogs or aerosols.

Liquid compositions, for example, in the form of emulsifiable or soluble concentrates most frequently comprise about 5 to about 80% by weight of the active ingredient, while the emulsions or solutions which are ready for application contain, in their case, about 0.01 to about 20% of the active ingredient. Besides the solvent, the emulsifiable or soluble concentrates may contain, when required, about 2 to about 50% of suitable additives, such as stabilizers, surface-active agents, penetrating agents, corrosion inhibitors, colorants or adhesives. Emulsions of any required concentration, which are particularly suitable for application, for example, to plants, may be obtained from these concentrates by dilution with water. These compositions are included within the scope of the compositions which may be employed in the present invention. The emulsions may be in the form of water-in-oil or oil-in-water type and they may have a thick consistency.

The liquid compositions of this invention may, in addition to normal agricultural use applications be used for example to treat substrates or sites infested or liable to infestation by arthropods (or other pests controlled by compounds of this invention) including premises, outdoor or indoor storage or processing areas, containers or equipment or standing or running water.

All these aqueous dispersions or emulsions or spraying mixtures can be applied, for example, to crops by any suitable means, chiefly by spraying, at rates which are generally of the order of about 100 to about 1,200 liters of spraying mixture per hectare, but may be higher or lower (eg. low or ultra-low volume) depending upon the need or application technique. The compounds or compositions according to the invention are conveniently applied to vegetation and in particular to roots or leaves having pests to be eliminated. Another method of application of the compounds or compositions according to the invention is by chemigation, that is to say, the addition of a formulation containing the active ingredient to irrigation water. This irrigation may be sprinkler irrigation for foliar pesticides or it can be ground irrigation or underground irrigation for soil or for systemic pesticides.

The concentrated suspensions, which can be applied by spraying, are prepared so as to produce a stable fluid product which does not settle (fine grinding) and usually contain from about 10 to about 75% by weight of active ingredient, from about 0.5 to about 30% of surface-active agents, from about 0.1 to about 10% of thixotropic agents, from about 0 to about 30% of suitable additives, such as anti-foaming agents, corrosion inhibitors, stabilizers, penetrating agents, adhesives and, as the carrier, water or an organic liquid in which the active ingredient is poorly soluble or insoluble Some organic solids or inorganic salts may be dissolved in the carrier to help prevent settling or as antifreezes for water.

The wettable powers (or powder for spraying) are usually prepared so that they contain from about 10 to about 80% by weight of active ingredient, from about 20 to about 90% of a solid carrier, from about 0 to about 5% of a wetting agent, from about 3 to about 10% of a dispersing agent and, when necessary, from about 0 to about 80% of one or more stabilizers and/or other additives, such as penetrating agents, adhesives, anti-caking agents, colorants, or the like. To obtain these wettable powders, the active ingredient(s) is(are) thoroughly mixed in a suitable blender with additional substances which may be impregnated on the porous filler and is(are) ground using a mill or other suitable grinder. This produces wettable powders, the wettability and the suspendability of which are advantageous. They may be suspended in water to give any desired concentration and this suspension can be employed very advantageously in particular for application to plant foliage.

The “water dispersible granules (WG)” (granules which are readily dispersible in water) have compositions which are substantially close to that of the wettable powders. They may be prepared by granulation of formulations described for the wettable powders, either by a wet route (contacting finely divided active ingredient with the inert filler and a little water, e.g. 1 to 20% by weight, or with an aqueous solution of a dispersing agent or binder, followed by drying and screening), or by a dry route (compacting followed by grinding and screening).

The rates and concentrations of the formulated compositions may vary according to the method of application or the nature of the compositions or use thereof. Generally speaking, the compositions for application to control arthropod, plant nematode, helminth or protozoan pests usually contain from about 0.00001% to about 95%, more particularly from about 0.0005% to about 50% by weight of one or more compounds of formula (I), or pesticidally acceptable salts thereof, or of total active ingredients (that is to say the compound of formula (I), or a pesticidally acceptable salt thereof, together with: other substances toxic to arthropods or plant nematodes, anthelmintics, anticoccidials, synergists, trace elements or stabilizers). The actual compositions employed and their rate of application will be selected to achieve the desired effect(s) by the farmer, livestock producer, medical or veterinary practitioner, pest control operator or other person skilled in the art.

Solid or liquid compositions for application topically to animals, timber, stored products or household goods usually contain from about 0.00005% to about 90%, more particularly from about 0.001% to about 10%, by weight of one or more compounds of formula (I) or pesticidally acceptable salts thereof. For administration to animals orally or parenterally, including percutaneously solid or liquid compositions, these normally contain from about 0. 1% to about 90% by weight of one or more compounds of formula (I) or pesticidally acceptable salts thereof. Medicated feedstuffs normally contain from about 0.001% to about 3% by weight of one or more compounds of formula (I) or pesticidally acceptable salts thereof. Concentrates or supplements for mixing with feedstuffs normally contain from about 5% to about 90%, preferably from about 5% to about 50%, by weight of one or more compounds of formula (I) or pesticidally acceptable salts thereof. Mineral salt licks normally contain from about 0.1% to about 10% by weight of one or more compounds of formula (I) or pesticidally acceptable salts thereof.

Dusts or liquid compositions for application to livestock, goods, premises or outdoor areas may contain from about 0.0001% to about 15%, more especially from about 0.005% to about 2.0%, by weight, of one or more compounds of formula (I) or pesticidally acceptable salts thereof. Suitable concentrations in treated waters are between about 0.0001 ppm and about 20 ppm, more particularly about 0.001 ppm to about 5.0 ppm. of one or more compounds of formula (I), or pesticidally acceptable salts thereof, and may be used therapeutically in fish farming with appropriate exposure times. Edible baits may contain from about 0.01% to about 5%, preferably from about 0.01% to about 1.0%, by weight, of one or more compounds of formula (I) or pesticidally acceptable salts thereof.

When administered to vertebrates parenterally, orally or by percutaneous or other means, the dosage of compounds of formula (I), or pesticidally acceptable salts thereof, will depend upon the species, age, or health of the vertebrate and upon the nature and degree of its actual or potential infestation by arthropod, helminth or protozoan pests. A single dose of about 0.1 to about 100 mg, preferably about 2.0 to about 20.0 mg, per kg body weight of the animal or doses of about 0.01 to about 20.0 mg, preferably about 0.1 to about 5.0 mg, per kg body weight of the animal per day, for sustained medication, are generally suitable by oral or parenteral administration. By use of sustained release formulations or devices, the daily doses required over a period of months may be combined and administered to animals on a single occasion.

The following composition EXAMPLES 2A -2M illustrate compositions for use against arthropods, especially mites or insects, plant nematodes, or helminth or protozoan pests which comprise, as active ingredient, compounds of formula (I), or pesticidally acceptable salts thereof, such as those described in preparative examples. The compositions described in EXAMPLES 2A -2M can each be diluted to give a sprayable compositon at concentrations suitable for use in the field. Generic chemical descriptions of the ingredients (for which all of the following percentages are in weight percent), used in the composition EXAMPLES 2A -2M exemplified below, are as follows:

| Trade Name | Chemical Description |
|---|---|
| Ethylan BCP | Nonylphenol ethylene oxide condensate |
| Soprophor BSU | Tristyrylphenol ethylene oxide condensate |
| Arylan CA | A 70% w/v solution of calcium dodecylbenzene- |
| | sulfonate |
| Solvesso 150 | Light C₁₀ aromatic solvent |
| Arylan S | Sodium dodecylbenzenesulfonate |
| Darvan No2 | Sodium lignosulphonate |
| Celite PF | Synthetic magnesium silicate carrier |
| Sopropon T36 | Sodium salts of polycarboxylic acids |
| Rhodigel 23 | Polysaccharide xanthan gum |
| Bentone 38 | Organic derivative of magnesium montmorillonite |
| Aerosil | Microfine silicon dioxide |

### EXAMPLE 2A

A water soluble concentrate is prepared with the composition as follows:

| | |
|---|---|
| Active ingredient | 7% |
| Ethylan BCP | 10% |
| N-methylpyrrolidone | 83% |

To a solution of Ethylan BCP dissolved in a portion of N-methylpyrrolidone is added the active ingredient with heating and stirring until dissolved. The resulting solution is made up to volume with the remainder of the solvent.

### EXAMPLE 2B

An emulsifiable concentrate (EC) is prepared with the composition as follows:

| Active ingredient | 25% (max) |
|---|---|
| Soprophor BSU | 10% |
| Arylan CA | 5% |
| N-methylpyrrolidone | 50% |
| Solvesso 150 | 10% |

The first three components are dissolved in N-methylpyrrolidone and to this is then added the Solvesso 150 to give the final volume.

### EXAMPLE 2C

A wettable powder (WP) is prepared with the composition as follows:

| | |
|---|---|
| Active ingredient | 40% |
| Arylan S | 2% |
| Darvan No2 | 5% |
| Celite PF | 53% |

The ingredients are mixed and ground in a hammer-mill to a powder with a particle size of less than 50 microns.

### EXAMPLE 2D

An aqueous-flowable formulation is prepared with the composition as follows:

| | |
|---|---|
| Active ingredient | 40.00% |
| Ethylan BCP | 1.00% |
| Sopropon T360. | 0.20% |
| Ethylene glycol | 5.00% |
| Rhodigel 230. | 0.15% |
| Water | 53.65% |

The ingredients are intimately mixed and are ground in a bead mill until a mean particle size of less than 3 microns is obtained.

### EXAMPLE 2E

An emulsifiable suspension concentrate is prepared with the composition as follows:

| | |
|---|---|
| Active ingredient | 30.0% |
| Ethylan BCP | 10.0% |
| Bentone 38 | 0.5% |
| Solvesso 150 | 59.5% |

The ingredients are intimately mixed and ground in a beadmill until a mean particle size of less than 3 microns is obtained.

### EXAMPLE 2F

A water dispersible granule is prepared with the composition as follows:

| | |
|---|---|
| Active ingredient | 30% |
| Darvan No2 | 15% |
| Arylan S | 8% |
| Celite PF | 47% |

The ingredients are mixed, micronized in a fluid-energy mill and then granulated in a rotating pelletizer by spraying with water (up to 10%). The resulting granules are dried in a fluid-bed drier to remove excess water.

### EXAMPLE 2G

A dusting powder is prepared with the composition as follows:

| | |
|---|---|
| Active ingredient | 1 to 10% |
| Talc powder-superfine | 99 to 90% |

The ingredients are intimately mixed and further ground as necessary to achieve a fine powder. This powder may be appplied to a locus of arthropod infestation, for example refuse dumps, stored products or household goods or animals infested by, or at risk of infestation by, arthropods to control the arthropods by oral ingestion. Suitable means for distributing the dusting powder to the locus of arthropod infestation include mechanical blowers, handshakers or livestock self treatment devices.

### EXAMPLE 2H

An edible bait is prepared with the composition as follows:

| | |
|---|---|
| Active ingredient | 0.1 to 1.0% |
| Wheat flour | 80% |
| Molasses | 19.9 to 19% |

The ingredients are intimately mixed and formed as required into a bait form. This edible bait may be distributed at a locus, for example domestic or industrial premises, e.g. kitchens, hospitals or stores, or outdoor areas, infested by arthropods, for example ants, locusts, cockroaches or flies, to control the arthropods by oral ingestion.

### EXAMPLE 2I

A solution formulation is prepared with a composition as follows:

| | |
|---|---|
| Active ingredient | 15% |
| Dimethyl sulfoxide | 85% |

The active ingredient is dissolved in dimethyl sulfoxide with mixing and or heating as required. This solution may be applied percutaneously as a pour-on application to domestic animals infested by arthropods or, after sterilization by filtration through a polytetrafluoroethylene membrane (0.22 micrometer pore size), by parenteral injection, at a rate of application of from 1.2 to 12 ml of solution per 100 kg of animal body weight.

### EXAMPLE 2J

A wettable powder is prepared with the composition as follows:

Active ingredient 50%

Ethylan BCP 5%

Aerosil 5%

Celite PF 40%

The Ethylan BCP is absorbed onto the Aerosil which is then mixed with the other ingredients and ground in a hammer-mill to give a wettable powder, which may be diluted with water to a concentration of from 0.001% to 2% by weight of the active compound and applied to a locus of infestation by arthropods, for example, dipterous larvae or plant nematodes, by spraying, or to domestic animals infested by, or at risk of infection by arthropods, helminths or protozoa, by spraying or dipping, or by oral administration in drinking water, to control the arthropods, helminths or protozoa.

### EXAMPLE 2K

A slow release bolus composition is formed from granules containing the following components in varying percentages(similar to those described for the previous compositions) depending upon need:

Active ingredient

Density agent

Slow-release agent

Binder

The intimately mixed ingredients are formed into granules which are compressed into a bolus with a specific gravity of 2 or more. This can be administered orally to ruminant domestic animals for retention within the reticulo-rumen to give a continual slow release of active compound over an extended period of time to control infestation of the ruminant domestic animals by arthropods, helminths or protozoa.

### EXAMPLE 2L

A slow release composition in the form of granules, pellets, brickettes or the like can be prepared with compositions as follows:

| | |
|---|---|
| Active ingredient | 0.5 to 25% |
| Polyvinyl chloride | 75 to 99.5% |

Dioctyl phthalate (plasticizer)

The components are blended and then formed into suitable shapes by melt-extrusion or molding. These composition are useful, for example, for addition to standing water or for fabrication into collars or eartags for attachment to domestic animals to control pests by slow release.

### EXAMPLE 2M

A water dispersible granule is prepared with the composition as follows:

| Active ingredient | 85% (max) |
|---|---|
| Polyvinylpyrrolidone | 5% |
| Attapulgite clay | 6% |
| Sodium lauryl sulfate | 2% |
| Glycerine | 2% |

The ingredients are mixed as a 45% slurry with water and wet milled to a particle size of 4 microns, then spray-dried to remove water.

While the invention has been described in terms of various preferred embodiments, the skilled artisan will appreciate that various modifications, substitutions, omissions and changes can be made without departing from the spirit thereof. Accordingly, it is intended that the scope of the present invention be limited solely by the scope of the following claims, including equivalents thereof.

What is claimed is:

1. A compound having the formula: 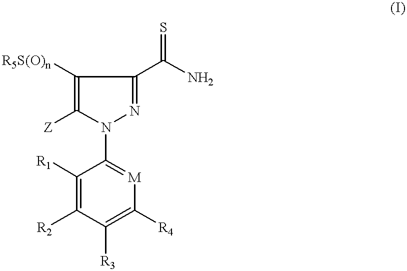
or a pesticidally acceptable salt thereof, wherein:
R₁ is H or halogen;
each of R₂ and R₄, which are identical or different, is H, halogen or alkyl;
R₃ is halogen, haloalkyl, haloalkoxy or R₁₀S(O)ₘ;
R₅ is lower alkyl, haloalkyl, alkenyl or alkynyl, or a cycloalkyl ring having 3 to 5 carbon atoms;
Z is hydrogen, halogen, alkyl, formyl, —C(O)alkyl, haloalkyl, alkenyl, hydrazino, alkoxycarbonyl, alkylthiocarbonyl, alkoxyalkylideneamino, 1H-pyrrol-1-yl, 1H-pyrazol-1-yl, amino, R₆NH— or R₇R₈N—;
each of R₆, R₇ and R₈, which are identical or different, is alkyl—S(O)P—, formyl, alkynyl having from 3 to 6 carbon atoms, alkoxycarbonyl, alkylthiocarbonyl or aroyl; or alkyl, alkenyl having 3 to 6 carbon atoms, or —C(O)alkyl wherein the alkyl and alkenyl portions are optionally substituted by one or more R₉;
or R₇ and R₈ are joined together so as to form a divalent radical having 4 to 6 atoms in the chain, this divalent radical being alkylene, alkyleneoxyalkylene or alkyleneaminoalkylene;
R₉ is cyano, nitro, alkoxy, haloalkoxy, R₁₀S(O)_{q}, —C(O)alkyl, alkoxycarbonyl, aminocarbonyl, alkylaminocarbonyl, dialkylaminocarbonyl, —CO₂H, halogen, hydroxy, aminosulfonyl, alkylaminosulfonyl or dialkylaminosulfonyl;
R₁₀ is lower alkyl or lower haloalkyl;
m, n, p and q are 0, 1 or 2; and
M is C-halo, C—CH₃, C—CH₂F, C—CH₂Cl, C—NO₂ or N.

2. A compound according to claim 1, wherein R₃ is halogen, haloalkyl or haloalkoxy.

3. A compound according to claim 1, wherein R₅ is alkyl.

4. A compound according to claim 1, wherein R₇ and R₈ are joined together so as to form a morpholine, pyrrolidine, piperidine or piperazine ring.

5. A compound according to claim 1, wherein Z is amino, R₇NH— or R₈R₉N—.

6. A compound according to claim 2, wherein R₅ is alkyl.

7. A compound according to claim 2, wherein Z is amino, R₇NH— or R₈R₉N—.

8. A compound according to claim 3, wherein Z is amino, R₇NH— or R₈R₉N—.

9. A compound according to claim 6, wherein Z is amino, R₇NH— or R₈R₉N—.

10. A compound according to claim 1, wherein:
R₁ is F, Cl, Br or H;
R₂and R₄are H;
R₃ is —CF₃, —OCF₃, —CHF₂, —S(O)ₘCF₃, —CFCl₂, —CF₂Cl, —OCF₂Cl, —OCFCl₂, Cl, Br or F;
R₅is methyl or ethyl;
Z is H, F, Cl, Br, C₁-C₃ alkyl, C₁-C₃ haloalkyl, or C₂-C₃ alkenyl; or Z is amino, —NHR₆ or —NR₇R₈, wherein each of R₆, R₇ and R₈, which are identical or different, is C₁-C₃ alkyl, C₃ alkenyl or —C(O)alkyl wherein the alkyl or alkenyl portions are unsubstituted or substituted with cyano, alkoxy, alkyl—S(O)ₚ—, nitro, alkoxycarbonyl, —C(O)alkyl, aminocarbonyl, alkylaminocarbonyl, dialkylaminocarbonyl, —CO 2H, hydroxy, F, Cl, or Br; and
M is CCl, CF, CBr or N.

11. A compound according to claim 1, wherein:
R₁ is Cl or Br;
R₂ and R₄ are H;
R₃ is —CF₃, —OCF₃ or Cl;
R₅ is methyl or ethyl;
Z is H, F, Cl, Br, C₁-C₃ alkyl, C₁-C₃ haloalkyl, C₂-C₃ alkenyl; or Z is amino, —NHR₆ or —NR₇R₈, wherein each of R₆, R₇ and R₈, which are identical or different, is C₁-C₃ alkyl, C₃ alkenyl or —C(O)alkyl wherein the alkyl or alkenyl portions are unsubstituted or substituted with cyano, alkoxy, alkyl-S(O) p nitro, alkoxycarbonyl, —C(O)alkyl, aminocarbonyl, alkylaminocarbonyl, dialkylaminocarbonyl, —CO ₂H, hydroxy, F, Cl, or Br; and
M is CCl, CBr or N.

12. A compound according to claim 1, wherein:
R₁ is Cl or Br;
R₂ and R₄ are H;
R₃ is —CF₃, —OCF₃ or Cl;
R₅ is optionally halogenated methyl or ethyl;
Z is H or amino; and
M is CCl, CBr or N.

13. A compound according to claim 1, wherein:
R₁ is Cl or Br;
R₂ and R₄ are H;
R₃ is —CF₃, —OCF₃ or Cl;
R₅ is methyl or ethyl;
Z is H or amino; and
M is CCl, CBr or N.

14. A compound according to claim 1, wherein the 1-aryl group is: 2,6-dichloro-4-trifluoromethylphenyl; 2,6-dichloro-4-trifluoromethoxyphenyl; 2-bromo-6-chloro-4-trifluoromethylphenyl; 2-bromo-6-chloro-4-trifluoromethoxyphenyl; 2,6-difluoro-4-trifluoromethylphenyl; 2-chloro-4-trifluoromethylphenyl; 2,6-dichloro-3-methyl-4-trifluoromethylphenyl; 3-chloro-5-trifluoromethyl-2-pyridinyl; 3-chloro-5-trifluoromethoxy-2-pyridinyl; 3,5-dichloro-2-pyridinyl; 2,6-dichloro-4-bromophenyl; 2,4,6-trichlorophenyl; 2-bromo-6-fluoro-4-difluoromethylphenyl; 2-chloro-6-fluoro-4-rifluoromethylphenyl; 2,6-dibromo-4-trifluoromethylphenyl; 2,6-dibromo-4-rifluoromethoxyphenyl; or 2-bromo-4-trifluoromethylphenyl.

15. A compound according to claim 1, wherein Z is acetylamino, amino, 2-n-butoxypropionylamino, methyl, hydroxyacetylamino, ethyl, 3-ethylsulfinylpropylamino, bromo, formylamino, chloro, methylamino, ethylamino, 2-hydroxyethylamino, 2-methoxyethylamino, methylsulfonylamino, 2-ethylsulfonylethylamino, 4-methoxybenzoylamino, 2-cyanoethylamino, 4-methoxybenzylamino, 2-methylthioethylamino, 2-aminocarbonylethylamino, 2-methylsulfinylethylamino, 3-methoxycarbonylpropylamino, 2-ethylsulfinylethylamino, 2-methylsulfonylethylamino, cyanomethylamino, 2-ethylthioethylamino, aminocarbonylmethylamino, dimethylamino, 2-nitroethylamino, 2-acetylethylamino, methylcarbonylmethylamino, methoxycarbonylamino, or ethoxycarbonylamino.

16. A compound according to claim 1, wherein —S(O)ₙR₅ is methylthio, methylsulfinyl, methylsulfonyl, ethylsulfinyl, ethylsulfonyl, ethylthio, cyclopropylsulfinyl, cyclopropylthio, cyclopropylsulfonyl, isopropylsulfinyl, isopropylsulfonyl or isopropylthio.

17. The compound according to claim 1, which is:
5-amino-1-(2,6-dichloro-4-rifluoromethylphenyl)-4-methylthiopyrazole-3-thiocarboxamide;
5-amino-1-(2,6-dichloro-4-trifluoromethylphenyl)-4-trifluoromethylthiopyrazole-3-thiocarboxamide;
4-dichlorofluoromethylsulfonyl-1-(2,4,6-trichlorophenyl)pyrazole-3-thiocarboxamide;
5-amino-1-(2,6-dichloro-4-trifluoromethylphenyl)-4-methylsulfinylpyrazole-3-thiocarboxamide;
5-amino-1-(2,6-dichloro-4-trifluoromethylphenyl)-4-methylsulfonylpyrazole-3-thiocarboxamide;
5-amino-1-(2-bromo-6-chloro-4-trifluoromethylphenyl)-4-methylsulfinylpyrazole-3-thiocarboxamide;
5-amino-1-(2,6-dichloro-4-trifluoromethoxyphenyl)-4-methylsulfinylpyrazole-3-thiocarboxamide;
5-amino-1-(2,6-dichloro-4-trifluoromethylphenyl)-4-ethylsulfinylpyrazole-3-thiocarboxamide;
5-amino-1-(2-chloro-4-trifluoromethylphenyl)-4-methylsulfinylpyrazole-3-thiocarboxamide;
5-amino-1-(2-bromo-6-chloro-4-trifluoromethylphenyl)-4-ethylsulfinylpyrazole-3-thiocarboxamide; or
5-amino-1-(2,6-dichloro-4-trifluoromethoxyphenyl)-4-ethylsulfmylpyrazole-3-thiocarboxamide.

18. A pesticidal composition comprising a pesticidally effective amount of a compound of formula (I) according to claim 1 and an agriculturally acceptable inert carrier thereof.

19. A pesticidal composition according to claim 18 which contains from about 0.05 to about 95% by weight of a compound of formula (I).

20. A pesticidal composition according to claim 18 which contains from about 0.00005 to about 90% by weight of a compound of formula (I).

21. A method for controlling pests at a locus comprising applying to said locus a pesticidally effective amount of a compound of formula (I) according to claim 1.

22. The method according to claim 21, wherein said pests are insects.

23. The method according to claim 22, wherein said insects are sucking insects.

24. The method according to claim 21, wherein said locus is a crop area.

25. The method according to claim 21, wherein said compound is applied to said locus at a rate of from about 5 g to about 1 kg/ha.

26. The method according to claim 21, wherein said locus is an animal.

27. The method according to claim 26, wherein said compound is applied to said locus at a rate of from about 0.1 to 20 mg per kg body weight of the animal per day.

28. A method for controlling pests at a locus comprising applying to said locus a pesticidally effective amount of a composition according to claim 18.

29. The method according to claim 28, wherein said pests are insects.

30. The method according to claim 29, wherein said insects are sucking insects.

31. The method according to claim 28, wherein said locus is a crop area.

32. The method according to claim 28, wherein said composition is applied to said locus at a rate of from about 5 g to about 1 kg of compound of formula (I) per hectare.

33. The method according to claim 28, wherein said locus is an animal.

34. The method according to claim 33, wherein said composition is applied to said locus at a rate of from about 0.1 to 20 mg of compound of formula (I) per kg body weight of the animal per day.

35. A process for preparing a compound of formula (I) as defined in claim 1 which comprises:
(a) reacting a compound having the formula: 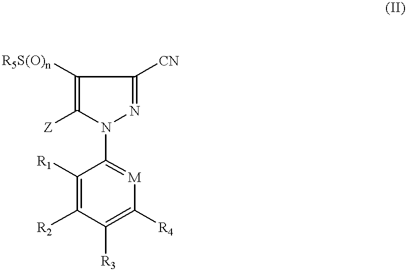
wherein R₁, R₂, R₃, R₄, R₅, M and Z are as defined in claim 1, with an alkali or alkaline earth metal hydrosulfide, to afford the corresponding compound of formula (I);
(b) oxidizing a compound of formula (I) wherein R₁, R₂, R₃, R₄, R₅, M and Z are as defined in claim 1 and n is 0 or 1, to afford the corresponding compound of formula (I) wherein n is 1 or 2;
(c) alkylating a compound of formula (I) wherein R₁, R₂, R₃, R₄, R₅ and M are as defined in claim 1 and Z is amino, with a compound of the formula:
R₉—CH═CH₂ (II)
wherein R₉ is as defined in claim 1, to afford the corresponding compound of formula (I) wherein Z is R₆NH— wherein R₆ is ethyl substituted at the 2-position by R₉, wherein R₉ is cyano, nitro, —S(O)_{q}R₁₀, —C(O)alkyl, alkoxycarbonyl, alkylaminocarbonyl, dialkylaminocarbonyl, aminosulfonyl, alkylaminosulfonyl or dialkylaminosulfonyl;
(d) alkylating a compound of formula (I) wherein R₁, R₂, R₃, R₄, R₅ and M are as defined in claim 1 and Z is amino, with a compound of the formula:
R₁₁—Y (IV)
wherein R₁₁, is alkyl or haloalkyl optionally substituted by one or more R₉ and Y represents a leaving group, to afford the corresponding compound of formula (I) wherein Z is —NHR₆ or —NR₇R₈ wherein R₆, R₇ and R₈ are alkyl or haloalkyl optionally substituted by one or more R₉;
(e) esterifying a compound of formula (I) wherein Z is carboxy with an alcohol of the formula:
R₁₂OH (V)
wherein R₁₂ is alky, to afford the corresponding compound of formula (I) wherein Z is alkoxycarbonyl;
(f) esterifying a compound of formula (I) wherein Z is carboxy with a thiol of the formula:
R₁₂SH (VI)
wherein R₁₂ is alkyl, to afford the corresponding compound of formula (I) wherein Z is alkylthiocarbonyl;
(g) diazotizing a compound of formula (I) wherein Z is amino, followed by reacting the resultant compound with a compound of the formula:
R₁₃CH═CH₂ (VII)
in the presence of a copper salt, to afford the corresponding compound of formula (I) wherein Z is R₁₃CH(Cl)CH₂— wherein R₁₃ is alkyl;
(h) oxidizing a compound of the formula: 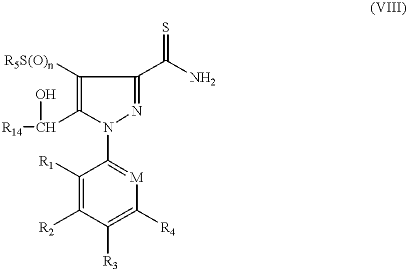
wherein R₁, R₂, R₃, R₄, R₅and M are as defined in claim 1 and R₁₄represents alkyl, to afford the corresponding compound of formula (I) wherein Z is C(O)alkyl; or
(i) dehydrochlorinating a compound of formula (I) wherein Z is R₁₃CH(Cl)CH₂— wherein R₁₃ is alkyl, to afford the corresponding compound of formula (I) wherein Z is R₁₃CH═CH;
optionally followed by converting the compound of formula (I) thus obtained into a pesticidally acceptable salt thereof.
